(19) United States
(12) Patent Application Publication
(10) Pub. No.: US 2004/0077952 A1
(43) Pub. Date: Apr. 22, 2004

(54) System and method for improved diagnostic image displays

(76) Inventors: Patrick G. Rafter, Windham, NH (US); Mario Gutierrez, Wakefield, MA (US); Marc C. Filerman, Arlington, MA (US); Patrick D. DiNino, Everett, MA (US)

Correspondence Address: PHILIPS INTELLECTUAL PROPERTY & STANDARDS, P.O. BOX 3001, BRIARCLIFF MANOR, NY, 10510, US

(21) Appl. No.: 10/274,612

(22) Filed: Oct. 21, 2002

(51) Int. Cl. G06F 17/60; A61B 5/02

(52) U.S. Cl. 600/481; 705/3

(57) ABSTRACT

An improved ultrasound-imaging diagnostic system comprises a patient interface configured to measure a patient condition, an ultrasound-imaging system configured to obtain a plurality of medical diagnostic images of a patient treated with a contrast agent over time, a medical diagnostic image manager configured to associate at least one imaging parameter and the patient condition with each of the plurality of medical diagnostic images, and an operator interface configured to receive an operator preference for spatially arranging a plurality of medical diagnostic images. A method for arranging a plurality of diagnostic images comprises collecting a plurality of diagnostic images of a patient, wherein each of the diagnostic images is associated with an image-acquisition mode and a patient condition, receiving a diagnostic directive comprising information responsive to a diagnostician's preference to observe diagnostic images associated with an image-identifier selected from the group consisting of image-acquisition orientation, image-acquisition mode, and patient condition, identifying a subset of the plurality of diagnostic images responsive to the diagnostic directive, and forwarding the subset of the plurality of diagnostic images to an output device.

### DESCRIPTION OF THE RELATED ART

The human body is composed of tissues that are generally opaque. In the past, exploratory surgery was one common way to look inside the body. Today, doctors can use a vast array of imaging methods to obtain information about a patient. Some non-invasive imaging techniques include modalities such as X-ray, magnetic resonance imaging (MRI), computer-aided tomography (CAT), ultrasound, and so on. Each of these techniques has advantages that make it useful for observing certain medical conditions and parts of the body. The use of a specific test, or a combination of tests, depends upon the patient's symptoms and the disease being diagnosed.

Generally, a trained technician performs a number of tasks to record information required to diagnose one or more medical conditions using a diagnostic imaging system. The technician collects and may even edit portions of the recorded information to identify reference points in the anatomy. Regardless of the underlying image acquisition modality, the images may be recorded on videotape, fixed disk drives, or other data storage devices for later analysis by a physician. For example, images acquired and recorded during an ultrasound exam may be exported to a networked storage device and saved for later evaluation.

Many clinical diagnostic imaging studies are recorded as a particular test or tests are performed on a patient of interest by a technician. Generally, a trained technician performs a number of tasks in order to record information that is required to diagnose one or more medical conditions using an imaging acquisition system. The technician collects, and may even edit, portions of the recorded information or study to identify reference points in the patient's anatomy. The images can be recorded on videotape, fixed disk drives, as well as, other data storage devices for later analysis and reporting by a physician.

Ultrasound-imaging systems can create two-dimensional brightness or B-mode images of tissue in which the brightness of a pixel is based on the intensity of the received ultrasound echoes. In another common imaging modality, typically known as color-flow imaging, the flow of blood or movement of tissue is observed. Color-flow imaging takes advantage of the Doppler effect to color-encode image displays. In color-flow imaging, the frequency shift of backscattered-ultrasound waves is used to measure the velocity of the backscatterers from tissues or blood. The frequency of sound waves reflecting from the inside of blood vessels, heart cavities, etc. is shifted in proportion to the velocity of the blood cells. The frequency of ultrasound waves reflected from cells moving towards the transducer is positively shifted. Conversely, the frequency of ultrasound reflections from cells moving away from the transducer is negatively shifted. The Doppler shift may be displayed using different colors to represent speed and direction of flow. To assist diagnosticians and operators, the color-flow image may be superimposed on the B-mode image.

Ultrasound imaging can be particularly effective when used in conjunction with contrast agents. In contrast-agent imaging, gas or fluid filled micro-sphere contrast agents known as microbubbles are typically injected into a medium, normally the bloodstream. Due to their physical characteristics, contrast agents stand out in ultrasound examinations and therefore can be used as markers that identify the amount of blood flowing to or through the observed tissue. In particular, the contrast agents resonate in the presence of ultrasound fields producing radial oscillations that can be easily detected and imaged. Normally, this response is imaged at the second harmonic, 2fₜ of the fundamental or transmit frequency,fₜ. By observing anatomical structures after introducing contrast agents, medical personnel can significantly enhance imaging capability for diagnosing the health of blood-filled tissues and blood-flow dynamics within a patient's circulatory system. For example, contrast agent imaging is especially effective in detecting myocardial boundaries, assessing micro-vascular blood flow, and detecting myocardial perfusion.

Since the United States Food and Drug Administration (U.S.F.D.A.) approved Left Ventricular Opacification in January of 1998 for human diagnostic imaging, the use of ultrasound contrast agents during stress echocardiographic examinations has seen a steady increase. Imaging techniques are also improving to the point where myocardial opacification may soon become a reality.

Stress echocardiographic examinations are typically administered by observing a series of ultrasound images recorded while a patient is exercising on a treadmill, stationary bicycle, or other exercise apparatus. Patients that are unable to attain and sustain a desired heart rate via exercise for the duration of the examination may be treated with one or more pharmaceutical agents to elevate their heart rate or as in the case of perfusion, vasodilators to increase blood flow. Because it is undesirable to submit patients to these diagnostic conditions for an extended length of time, there is a desire to keep the acquisition time, and thus the examination times as short as possible. Although contrast agent imaging techniques increase the quality of the diagnostic images, the techniques can add significantly to the length of the examination and the volume of data that needs to be reviewed and analyzed after the data is collected. Consequently, there is a desire to minimize the time required for image acquisition and interpretation.

Some ultrasound-imaging systems include features, which enable viewing of clinical data along with images acquired during a stress echo examination. For example, the SONOS 5500, commercially available from Koninklijke Philips Electronics N.V., doing business as, Philips Electronics North America Corporation of Tarrytown, N.Y., United States of America, has a feature, which sequences acquired images for tissue motion analysis. The ultrasound images can be displayed in three display modes. A first display mode groups images by a corresponding patient-stress stage (i.e., the images are grouped by stage). The second display mode groups images of the same view (i.e., the images are grouped by subject matter and orientation of the ultrasound transducer). A third display mode displays the images chronologically (i.e., in the sequence in which the images were acquired). A user-selected display mode associates the images into the appropriate group. The operator may then elect to display the grouped images.

The introduction of contrast agent imaging techniques, which enable physicians and or other diagnosticians to view many different forms of clinical observations in addition to tissue motion has complicated the process of grouping acquired images in a clinically relevant manner. Contrast agent imaging techniques permit the acquisition of data in multiple modes, each of which may provide information on one or more clinical parameters. For example, for a given view at each stage of patient stress, a heart wall motion image can be obtained with or without contrast agent enhanced imaging techniques. Contrast agent imaging techniques also enable the acquisition of real-time perfusion data with loops up to 20 beats or seconds long, triggered perfusion data with a series of frames acquired over the span of 30 seconds to one minute, real-time images with border (i.e., tissue motion) tracking for one or more cardiac cycles, coronary blood-flow data with pulsed wave (PW) Doppler, or 3D images of the cardiac anatomy in addition to many other qualitative and quantitative measurements. Often, the technician will acquire multiple image loops per stress stage and may even acquire multiple loops of the same anatomical view and the same imaging mode at slightly different angles. While the multiple image loops can be acquired and/or stored chronologically throughout the examination, it is very time consuming for a diagnostician to sort through the multiple image loops to determine which images should be analyzed in detail and in what order the acquired images should be reviewed. Often, with contrast agent enhanced imaging loops it is desirable to break up a long loop such as a 20-second loop of real-time tissue perfusion imaging techniques or a one minute acquisition of triggered perfusion images. It is also very time consuming and tedious for the diagnostician to select appropriate portions of these loops for comparison and analysis. With 3D images, it is important for the diagnostician to be able to break a 3D image into a series of 2D images for easier comparison.

### SUMMARY

An improved ultrasound-imaging diagnostic display system comprises a patient interface configured to measure a patient condition, an ultrasound-imaging system communicatively coupled to the patient interface configured to obtain a plurality of medical diagnostic images of a patient treated with a contrast agent over time, a medical diagnostic image manager configured to associate at least one imaging parameter and the patient condition with each of the plurality of medical diagnostic images, and an operator interface configured to receive an operator preference for spatially arranging a plurality of medical diagnostic images. Furthermore, the ultrasound-imaging diagnostic display system comprises an interface that enables the user to modify acquired loops by segmenting the loops, combining frames obtained from multiple loops, and displaying the image loops in a manner desired by the diagnostician. The system also comprises an image selector that enables the diagnostic display system to display multiple images acquired from nearly the same perspective of the same anatomical structures, under the same patient condition(s) and same image-acquisition parameters.

A method for arranging a plurality of diagnostic images, comprises collecting a plurality of diagnostic images of a patient, wherein each of the diagnostic images is associated with an image-acquisition mode and a patient condition, receiving a diagnostic directive comprising information responsive to a diagnostician's preference to observe diagnostic images associated with an image-identifier selected from the group consisting of image-acquisition orientation, image-acquisition mode, and patient condition, identifying a subset of the plurality of diagnostic images responsive to the diagnostic directive, and forwarding the subset of the plurality of diagnostic images to an output device.

### BRIEF DESCRIPTION OF THE DRAWINGS

A system and method for improved diagnostic-image displays are illustrated by way of example and not limited by the embodiments depicted in the following drawings. The components in the drawings are not necessarily to scale. Emphasis instead is placed upon clearly illustrating the principles of the present system and method. Moreover, in the drawings, like reference numerals designate corresponding parts throughout the several views.

FIG. 1 is a schematic diagram illustrating an embodiment of a diagnostic-imaging management system.

FIG. 2 is a functional block diagram illustrating an embodiment of the diagnostic image-acquisition system of FIG. 1.

FIG. 3A is a plot of a typical adult electrocardiogram that can be produced by the patient condition sensor of FIG. 1.

FIG. 3B is a plot of patient-under-test stress over time that can be derived by the diagnostic image acquisition system of FIG. 2.

FIG. 4 is a functional block diagram illustrating an embodiment of the workstation of FIG. 1.

FIG. 5 is a schematic diagram illustrating an embodiment of a diagnostic image file that can be found in the data store of the image-management system of FIG. 1.

FIG. 6 is a functional block diagram illustrating an embodiment of an image manager application that can be stored and executed on the workstation of FIG. 4.

FIGS. 7A-7D present example embodiments of a diagnostic image display that can be produced on the workstation of FIG. 4.

FIG. 8 is a flow chart illustrating a method for improved diagnostic image displays that may be implemented by the diagnostic image-management system of FIG. 1.

### DETAILED DESCRIPTION

The present disclosure generally relates to a system and method for controllably arranging a plurality of diagnostic images. An operator of a diagnostic-image-management system uses an interface to define one or more preferred arrangements for displaying a plurality of diagnostic images on an output device. The operator defines the arrangements by associating a relative output-device position and image size with an image acquired under specific patient conditions and imaging parameters. Thereafter, the diagnostic-image-management system is programmed to identify and render a plurality of diagnostic images in accordance with the operator's preferences for observing the images.

An improved diagnostic-image-management system having been summarized above, reference will now be made in detail to the description of the system and method as illustrated in the drawings. For clarity of presentation, the diagnostic-image-management system (DIMS) and an embodiment of the underlying image manager will be exemplified and described with focus on the generation of a composite representation of diagnostic images in formats preferred by a diagnostician operator of the DIMS.

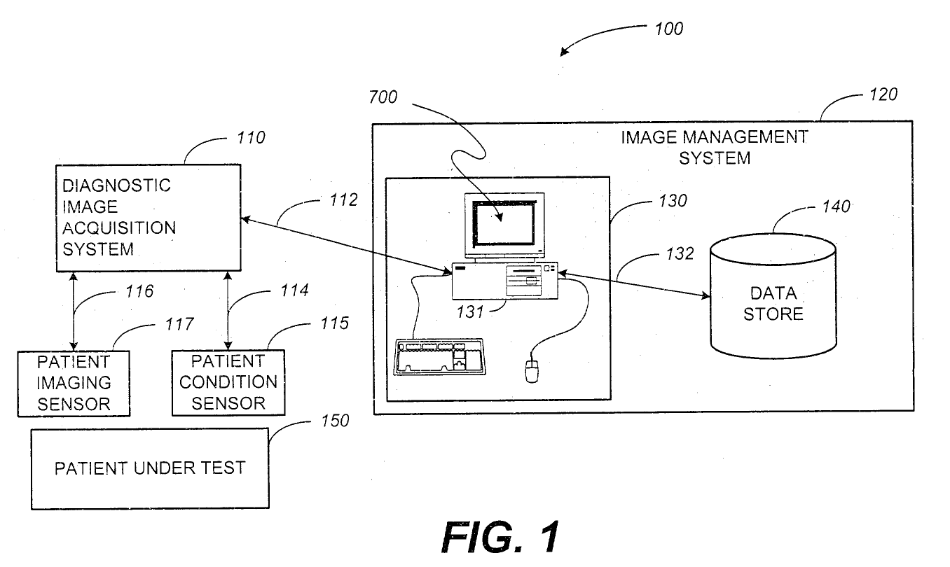

Turning now to the drawings, wherein like reference numerals designate corresponding parts throughout the drawings, reference is made to FIG. 1, which illustrates a schematic of an embodiment of a DIMS 100. As illustrated in the schematic of FIG. 1, DIMS 100 includes a diagnostic image-acquisition system 110 as well as an image-management system 120. Image-management system 120 includes workstation 130 and data store 140. Workstation 130 is communicatively coupled with data store 140 via interface 132.

Diagnostic image-acquisition system 110 and image-management system 120 are communicatively coupled to each other via interface 112 to enable an operator of workstation 130 to access, arrange, and display diagnostic images accumulated during one or more patient examinations. Diagnostic image-acquisition system 110 is coupled to patient condition sensor 115 and patient imaging sensor 117 via interface 114 and interface 116, respectively. Patient condition sensor 115 is configured to monitor one or more patient parameters or conditions, such as heart rate, respiratory rate, blood oxygen saturation, temperature, etc. Interface 114 is configured to communicatively couple one or more time varying signals from one or more transducers included within patient condition sensor 115 to the diagnostic image-acquisition system 110.

As will be explained below, a diagnostic image can be acquired by the diagnostic image acquisition system 110, or otherwise received by, the general-purpose computer 131 operating within the DIMS 100. For example, a diagnostic image can be acquired from an ultrasound imaging system, a computer-aided tomography (CAT) imaging system, a magnetic resonance imaging (MRI) system, among others.

Because the examples presented below describe heart studies of a patient-under-test 150 that include the acquisition, identification, and arrangement of ultrasound echo induced diagnostic images, subsequent references to patient condition sensor 115 are limited to transducers used in association with an electrocardiographic processor to produce a signal representative of heart muscle activity over time. However, patient-condition sensor 115 as used in the present system and method for improved diagnostic image displays is not limited to electrocardiographic transducers.

Patient imaging sensor 117 is configured to provide a plurality of signals via interface 116 to the diagnostic image-acquisition system 110. The plurality of signals are in turn received, buffered, and processed in accordance with known techniques in order to produce one or more graphic representations of various portions of the anatomy of the patient-under-test 150. In preferred embodiments, patient-imaging sensor 117 is an ultrasound transducer. In alternative embodiments, patient-imaging sensor 117 can include a magnetic resonance imaging sensor, an x-ray sensor, etc.

Workstation 130 includes a general-purpose computer 131. The general-purpose computer 131 is communicatively coupled to both data store 140 and diagnostic image-acquisition system 110 via interface 132 and interface 112, respectively. Interfaces 112, 132 can be wired interfaces, wireless (e.g., a radio-frequency) interfaces, and/or networks that couple workstation 130 to one or more diagnostic image-acquisition systems 110 and one or more distributed data storage devices included in data store 140. Alternatively, the image management system 120 can reside in the diagnostic image acquisition system 110.

Interfaces 112, 132 can be interfaces commonly available with general-purpose computers such as a serial, parallel, universal serial bus (USB), USB II, the institute of electrical and electronics engineers (IEEE) 1394 interface, also known as “Firewire®,” or the like. Firewire is the registered trademark of Apple Computer, Inc. of Cupertino, Calif., U.S.A. Furthermore, interfaces 112, 132 may use different standards or proprietary communications protocols for different types of image sources.

When interfaces 112, 132 are implemented via a network, the interfaces 112, 132 can be any local area network (LAN) or wide area network (WAN). When configured as a LAN, the LAN can be configured as a ring network, a bus network, and/or a wireless-local network. When the interfaces 112, 132 are implemented over a WAN, the WAN could be the public-switched telephone network, a proprietary network, and/or the public access WAN commonly known as the Internet.

Regardless of the actual network infrastructure used in particular embodiments, diagnostic-image data can be exchanged with general-purpose computer 131 of workstation 130 using various communication protocols. For example, transmission-control protocol/Internet protocol (TCP/IP) may be used if the interfaces 112, 132 are configured over a LAN or a WAN. Proprietary data-communication protocols may also be used when the interfaces 112, 132 are configured over a proprietary LAN or WAN.

Regardless of the underlying patient imaging technology used by the diagnostic image-acquisition system 110, images of the anatomy of the patient-under-test 150 are captured or otherwise acquired by an image-recording subsystem within the diagnostic image-acquisition system 110. Acquired images include information defining the characteristics observed for each of a plurality of picture elements or pixels that define the diagnostic image. Each pixel includes digital (i.e., numeric) information describing the colors and intensity of light observed at a particular region of an image sensor. The digital information arranged in a two-dimensional array of pixels can be used by suitably configured devices (e.g., the general-purpose computer 131, a photo-quality printer (not shown), etc.) to create a rendition of the captured image.

Because various types of image-processing devices can be easily coupled to the DIMS 100 (e.g., a video-tape recorder/player, a digital-video disk (DVD) recorder/player, etc.), previously recorded images stored on various media (e.g., a computer diskette, a flash-memory device, a compact-disk (CD), a magnetic tape, etc.) can be transferred to workstation 130 and/or data store 140 for processing in accordance with an image manager application program operable on the general-purpose computer 131 of the workstation 130. After processing by the image-management system 120 in accordance with preferred methods for arranging and displaying a plurality of the acquired and/or previously stored diagnostic images, the DIMS 100 can store the various composite image arrangements on a suitable data-storage medium.

Those skilled in the art will understand that a plurality of images from one or more patient studies can be presented in sequence. Such sequences or image loops can be repeated (i.e., the general-purpose computer 131 can present the first image and each subsequent image in the sequence after the last image in the sequence has been presented) as may be desired by a diagnostician or other operator of the image-management system 120.

Any combination of image-acquisition devices and/or data-storage devices may be included in DIMS 100. In addition, DIMS 100 may contain more than one image source of the same type. DIMS 100 may further include devices to which a digital image captured or otherwise acquired from a diagnostic image-acquisition system or a data-storage device can be sent. Such devices include hard-copy output devices such as a photo-quality printer.

Those skilled in the art will understand that various portions of DIMS 100 can be implemented in hardware, software, firmware, or combinations thereof. In a preferred embodiment, DIMS 100 is implemented using a combination of hardware and software or firmware that is stored in memory and executed by a suitable instruction-execution system. If implemented solely in hardware, as in an alternative embodiment, DIMS 100 can be implemented with any or a combination of technologies which are well-known in the art (e.g., discrete-logic circuits, application-specific integrated circuits (ASICs), programmable-gate arrays (PGAs), field-programmable gate arrays (FPGAs), etc.), or later developed technologies. In a preferred embodiment, the functions of the DIMS 100 are implemented in a combination of software and data executed and stored under the control of the general-purpose computer 131. It should be noted, however, that the DIMS 100 is not dependent upon the nature of the underlying computer in order to accomplish designated functions.

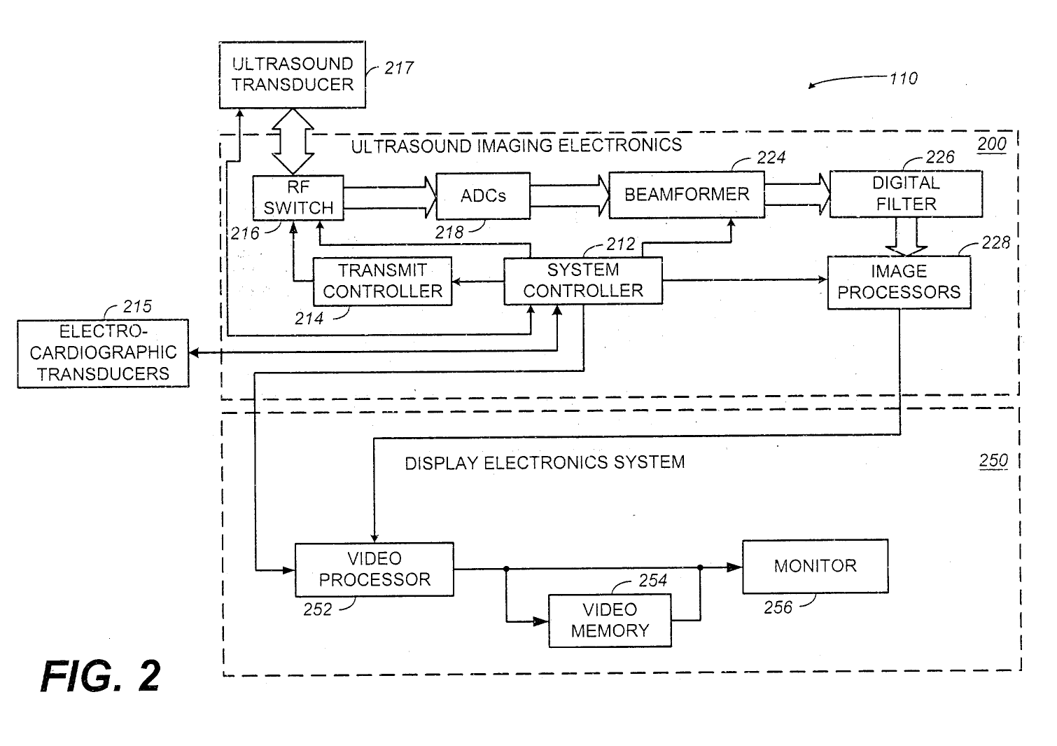

Reference is now directed to FIG. 2, which illustrates a functional block diagram of ant embodiment of the diagnostic image-acquisition system 110 of FIG. I. In this regard, the diagnostic image-acquisition system 110 may include ultrasound-imaging electronics 200 common to many ultrasound-imaging systems. As shown in FIG. 2, ultrasound-imaging electronics 200 are in communication with electrocardiographic transducer(s) 215, an ultrasound transducer 217, and a display electronics system 250. Ultrasound-imaging electronics 200 includes a system controller 212 that controls the operation and timing of the various functional elements and signal flows within the diagnostic image-acquisition system 110 pursuant to suitable software.

System controller 212 is coupled to transmit controller 214 which produces a plurality of various ultrasound signals that are controllably forwarded to the ultrasound transducer 217 via radio-frequency (RF) switch 216. Ultrasound echoes received from portions of the anatomy of the patient-under-test 150 (FIG. 1) are converted to electrical signals in ultrasound transducer 217 and forwarded via RF switch 216 to a receive channel that includes analog to digital converters 218, beamformer 224, digital filter 226, and various image processors 228.

Ultrasound transducer 217 is configured to emit and receive ultrasound signals, or acoustic energy, to and from an object-under-test (e.g., the anatomy of the patient-under-test) when the ultrasound-imaging electronics 200 are used in the context of a medical application). The ultrasound transducer 217 is preferably a phased-array transducer having a plurality of elements both in the azimuth and elevation directions.

In one embodiment, the ultrasound transducer 217 comprises an array of elements typically made of a piezoelectric material, for example but not limited to, lead-zirconate-titanate (PZT). Each element is supplied an electrical pulse or other suitable electrical waveform, causing the elements to collectively propagate an ultrasound-pressure wave into the object-under-test. Moreover, in response thereto, one or more echoes are reflected by various tissues within the patient and are received by the ultrasound transducer 217, which transforms the echoes into a plurality of electrical signals.

The array of elements associated with the ultrasound transducer 217 enable a beam, emanating from the transducer array, to be steered (during transmit and receive modes) through the patient-under-test by shifting the phase (introducing a time delay) of the electrical pulses (i.e., the transmit signals) supplied to the separate transducer elements. During a transmit mode, an analog waveform is communicated to each transducer element, thereby causing a pulse to be selectively propagated in a particular direction, like a beam, through the patient.

During a receive mode, an analog waveform is received at each transducer element at each transducer element. Each analog waveform essentially represents a succession of echoes received by the ultrasound transducer 217 over a period-of-time as echoes are received along the single beam through the patient. The entire set of analog waveforms represents an acoustic line, and the entire set of acoustic lines represents a single view, or image, of an object and is commonly referred to as a frame. Each frame represents a separate diagnostic image that can be stored within the image-management system 120 for later arrangement in a preferred diagnostic routine. Note that frame (i.e., image data storage) can be implemented on a frame by frame or a multiple frame basis.

In addition to forwarding the acquired digital images to image-management system 120, diagnostic image-acquisition system 10 can forward each image to display electronics systems 250. Display electronics system 250 includes video processor 252, video memory 254, and monitor 256. As shown in FIG. 2, monitor 256 may be configured to receive a video-input signal from video memory 254 and/or video processor 252. This multiple video signal input arrangement enables both real-time image observations, as well as post-test diagnostic viewing of stored diagnostic images. In order to enable post-test diagnostic viewing, video memory 254 can include a digital-video disk (DVD) player/recorder, a compact-disc (CD) player/recorder, a video-cassette recorder (VCR) or other various video-information storage devices.

Those skilled in the art will understand that display-electronics system 250 may be integrated and/or otherwise co-located with the diagnostic image-acquisition system 110. Alternatively, the display-electronics system 250 can be integrated and/or otherwise co-located with workstation 130. In other embodiments, separate display-electronics systems 250 can be integrated with workstation 130 and diagnostic image-acquisition system 110.

In operation, system controller 212 can be programmed or otherwise configured to forward one or more control signals to direct operation of the transmit, controller 214. Generally, a test technician will configure the ultrasound-imaging electronics 200 to coordinate the application of appropriate ultrasound signal transmissions, as well as to coordinate the selective observation of the resulting ultrasound echoes to record a plurality of image loops. Note that system controller 212 may forward various control signals in response to one or more signals received from electrocardiographic transducers 215 and/or other patient condition sensors (not shown). In response, transmit controller 214 generates a series of electrical pulses that are periodically communicated to a portion of the array of elements of the ultrasound transducer 217 via RF switch 216, causing the transducer elements to emit ultrasound signals into the object-under-test of the nature described previously. The transmit controller 214 typically provides separation (in time) between the pulsed transmissions to enable the ultrasound transducer 217 to receive echoes from patient-under-test tissues during the period between pulsed transmissions. RF switch 216 forwards the received echoes via the ADCs 218 to a set of parallel channels within the beamformer 224.

When the transmit pulses (in the form of ultrasound energy) encounter a tissue layer of the patient-under-test 150 that is receptive to ultrasound insonification, the multiple transmit pulses penetrate the tissue layer. As long as the magnitude of the multiple ultrasound pulses exceeds the attenuation affects of the tissue layer, the multiple ultrasound pulses will reach an internal target. Those skilled in the art will appreciate that tissue boundaries or intersections between tissues with different ultrasound impedances will develop ultrasound responses at the fundamental or transmit frequency,f₁, of the plurality of ultrasound pulses. Tissue insonified with ultrasound pulses will develop fundamental-ultrasound responses that may be distinguished in time from the transmit pulses to convey information from the various tissue boundaries within a patient.

Those ultrasound reflections of a magnitude that exceed that of the attenuation affects from traversing tissue layer may be monitored and converted into an electrical representation of the received ultrasound echoes. Those skilled in the art will appreciate that those tissue boundaries or intersections between tissues with different ultrasound impedances will develop ultrasound responses at both the fundamental frequency, fₜ, as well as, at harmonics (e.g., 2fₜ, 3fₜ, 4fₜ, etc.) of the fundamental frequency of the plurality of ultrasound pulses. Tissue insonified with ultrasound pulses will develop both fundamental and harmonic-ultrasound responses that may be distinguished in time from the transmit pulses to convey information from the various tissue boundaries within a patient. It will be further appreciated that tissue insonified with ultrasound pulses develops harmonic responses because the compressional portion of the insonified waveforms travels faster than the rarefactional portions. The different rates of travel of the compressional and the rarefactional portions of the waveform causes the wave to distort producing a harmonic signal, which is reflected or scattered back through the various tissue boundaries.

Preferably, ultrasound-imaging electronics 200 transmit a plurality of ultrasound pulses via ultrasound transducer 217 at a fundamental frequency and receive a plurality of ultrasound-echo pulses or receive pulses at an integer harmonic of the fundamental frequency. Those skilled in the art will appreciate that harmonic responses may be received by the same transducer when the ultrasound transducer 217 has an appropriately wide frequency band width.

While the internal target within the patient-under-test 150 will produce harmonic responses at integer multiples of the fundamental frequency, various contrast agents have been shown to produce subharmonic, harmonic, and ultraharmonic responses to incident ultrasound pulses. Consequently, observation of ultrasound echoes when the patient-under-test 150 has been treated (i.e., injected) with one or more contrast agents has proven beneficial to monitoring cardiac chambers, valves, and blood supply dynamics. Those ultrasound reflections of a magnitude that exceed that of the attenuation affects from traversing the various tissues of the patient-under-test 150 are converted into a plurality of electrical signal by the ultrasound transducer 217.

Beamformer 224 receives the echoes as a series of waveforms converted by ADCs 218. More specifically, beamformer 224 receives a digital version of an analog waveform from a corresponding transducer element for each acoustic line. Moreover, beamformer 224 receives a series of waveform sets, one set for each separate acoustic line, in succession over time and processes the waveforms in a pipeline-processing manner. Because the ultrasound signals received by ultrasound transducer 217 are of low power, a set of preamplifiers (not shown) may be disposed within beamformer 224.

In this way, beamformer 224 receives a series of waveforms corresponding to separate acoustic lines in succession over time and processes the data in a pipeline-processing manner. Beamformer 224 combines the series of received waveforms to form a single acoustic line. To accomplish this task, beamformer 224 may delay the separate echo waveforms by different amounts of time and then may add the delayed waveforms together, to create a composite digital RF acoustic line. The foregoing delay and sum beamforming process is well known in the art. Furthermore, beamformer 224 may receive a series of data collections for separate acoustic lines in succession over time and process the data in a pipeline-processing manner.

Because the echo waveforms typically decay in amplitude as they are received from progressively deeper depths in the patient, beamformer 224 may further comprise a parallel plurality of time-gain compensators (TGCs—not shown), which are designed to progressively increase the gain along the length of each acoustic line, thereby reducing the dynamic range requirements on subsequent processing stages. Moreover, the set of TGCs may receive a series of waveform sets, one set for each separate acoustic line, in succession over time and may process the waveforms in a pipeline-processing manner.

Each of the waveforms processed by beamformer 224 may be forwarded to digital filter 226. The waveforms include a number of discrete-location points (hundreds to thousands; corresponding with depth and ultrasound-transmit frequency) with respective quantized instantaneous signal levels, as is well known in the art. In previous ultrasound-imaging systems, this conversion often occurred later in the signal-processing stages, but recently, many of the logical functions that are performed on the ultrasonic signals can be digital, and hence, the conversion is preferred at an early stage in the signal-processing process.

Digital filter 226 can be configured as a frequency band pass filter configured to remove undesired high-frequency out-of-band noise from the plurality of waveforms. The output of the digital filter 226 can then be coupled to an I, Q demodulator (not shown) configured to receive and process digital-acoustic lines in succession. The I, Q demodulator may comprise a local oscillator that may be configured to mix the received digital-acoustic lines with a complex signal having an in-phase (real) signal and a quadrature-phase (imaginary) signal that are ninety degrees out-of-phase from one another. The mixing operation may produce sum and difference-frequency signals. The sum-frequency signal may be filtered (removed), leaving the difference-frequency signal, which is a complex signal centered near zero frequency. This complex signal is desired to follow direction of movement of anatomical structures imaged in the object-under-test, and to allow accurate, wide-bandwidth amplitude detection.

Up to this point in the ultrasound echo-receive process, all operations can be considered substantially linear, so that the order of operations may be rearranged while maintaining substantially equivalent function. For example, in some systems it may be desirable to mix to a lower intermediate frequency or to baseband before beamforming or filtering. Such rearrangements of substantially linear processing functions are considered to be within the skill set of those skilled in the art of ultrasound-imaging systems.

A plurality of signal processors 228 are coupled to the output of the digital filter 226 via I, Q demodulator. For example, a B-mode processor, a Doppler processor, and/or a color-flow processor, among others may be introduced at the output of the I, Q demodulator. Each of the image processors 228 includes a suitable species of random-access memory (RAM) and is configured to receive the filtered digital-acoustic lines. The acoustic lines can be defined within a two-dimensional coordinate space and may contain additional information that can be used in generating a three-dimensional image. Furthermore, the various image processors 228 accumulate acoustic lines of data over time for signal manipulation.

Regardless of the location of the display-electronics system 250, video processor 252 may be configured to produce two-dimensional and three-dimensional images from the data in the RAM once an entire data frame (i.e., a set of all acoustic lines in a single view or image to be displayed) has been accumulated by the RAM. For example, if the received data is stored in RAM using polar coordinates to define the relative location of the echo information, the video processor 252 may convert the polar coordinate data into rectangular (orthogonal) data capable of raster scan via a raster-scan capable display monitor 256.

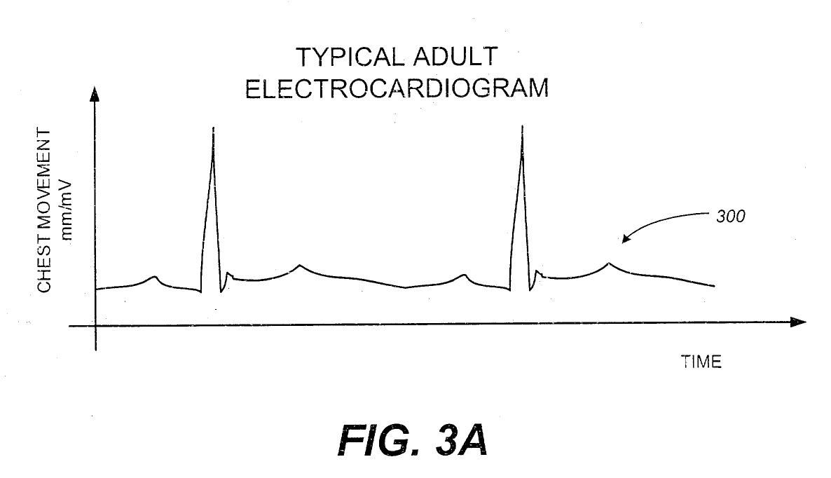

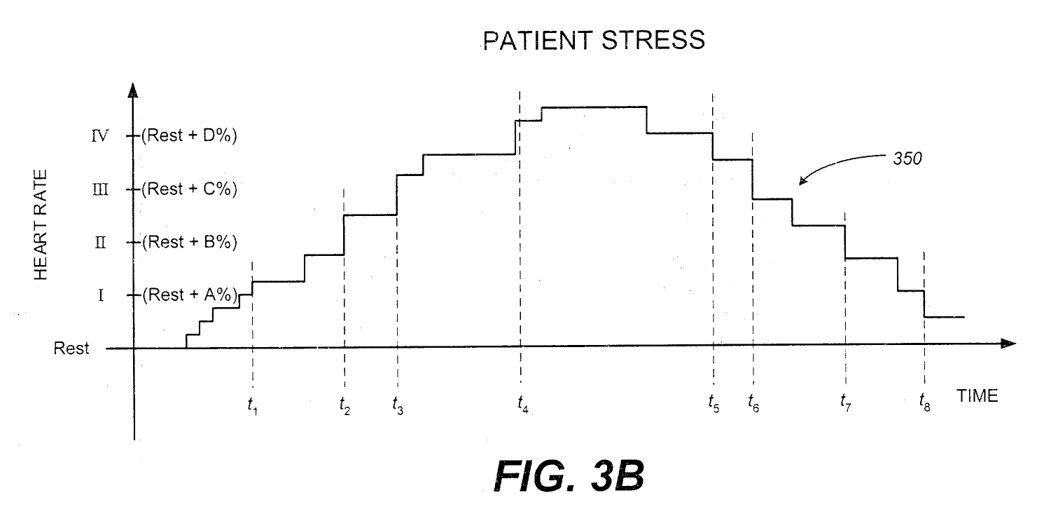

When patient-condition sensor 115 (FIG. 1) includes a plurality of electrocardiographic transducers 215 placed on the patient-under-test's chest, the plurality of transducers generate a set of electrical signals that represent chest movement over time. FIG. 3A illustrates a plot 300 of a typical adult's heart muscle activity (as observed through chest movement) over time as may be recorded by a suitably configured electrocardiographic-measurement subsystem within the diagnostic image-acquisition system 110 of FIG. 1. Because human heart motion is periodic, characteristic portions of the plot 300 can be used to trigger or otherwise coordinate the application of one or more transmit control signals via RF switch 216 to the ultrasound transducer 216 (FIG. 2). When the diagnostic image-acquisition system 110 is an ultrasound imaging system, ultrasound energy echoes received in the ultrasound transducer 217 as a result of transmitted ultrasound energy can be used to produce images that capture the heart muscle during specific events within the heart cycle. For example, one skilled in the art could use the plot 300 to coordinate the acquisition of an ultrasound image of the patient's heart that corresponds to the systole and diastole of the left ventricle. By coordinating the acquisition of multiple images of a patient's heart at a similar point in the heart cycle under multiple image-acquisition modes, viewing orientations, and patient conditions, a diagnostician can increase their understanding of the patient's condition.

FIG. 3B illustrates one way to quantify a patient's condition during a stress test. Stress tests are generally performed to give a diagnostician information regarding what is happening within a patient-under-test's heart when the patient's heart rate or blood flow increases. One way to quantify patient stress is to plot a patient's heart rate over time.

As illustrated in FIG. 3B, patient stress can be quantified in relation with a particular patient's heart rate at rest. Multiple stress stages can then be identified by applying a function to the patient's heart rate at rest. In the example of FIG. 3B, the patient achieves a stage I stress level when his heart rate increases by A, a predetermined percentage, above the patient's heart rate at rest. Stage II through stage IV stress levels are attained when the patient's heart rate exceeds the patient's heart rate at rest by larger percentages. As is further shown in FIG. 3B, the patient associated with patient stress plot 350 is characterized as attaining a stage I stress level during time periods t₁ to t₂ and t₇ to t₈. The patient attained stress level II during time periods t₂ to t₃ and t₆ to t₇. The patient attained stress level II during time periods t₃ to t₄ and t₅ to t₆. The patient attained the highest stress level, stress level IV, during time period t₄ to t₅. As will be further explained below, patient stress stage or stress level can be used as one of many patient conditions or patient parameters to enable a diagnostician of heart function to categorize, identify, and arrange a plurality of diagnostic images.

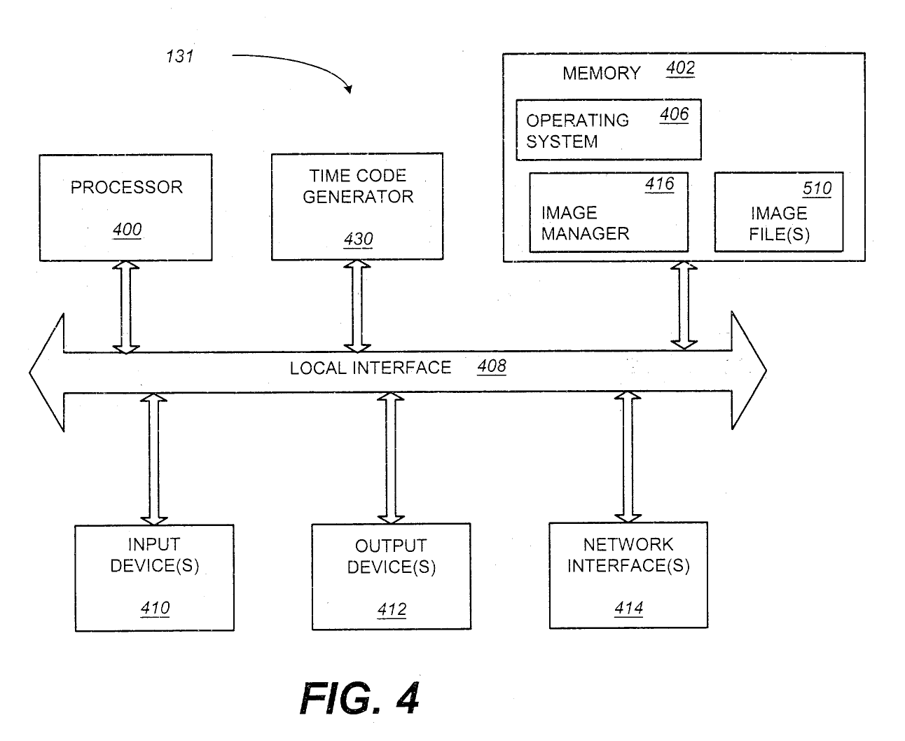

Reference is now directed to FIG. 4, which illustrates a functional block diagram of the general-purpose computer 131 of FIG. 1. Generally, in terms of hardware architecture, as shown in FIG. 4, the general-purpose computer 131 may include a processor 400, memory 402, input device(s) 410, output device(s) 412, and network interface(s) 414, that are communicatively coupled via local interface 408.

Local interface 408 can be, for example but not limited to, one or more buses or other wired or wireless connections, as is known in the art or may be later developed. Local interface 408 may have additional elements, which are omitted for simplicity, such as controllers, buffers (caches), drivers, repeaters, and receivers, to enable communications. Further, local interface 408 may include address, control, and/or data connections to enable appropriate communications among the aforementioned components of the general-purpose computer 131.

In the embodiment of FIG. 4, the processor 400 is a hardware device for executing software that can be stored in memory 402. The processor 400 can be any custom-made or commercially-available processor, a central-processing unit (CPU) or an auxiliary processor among several processors associated with the general-purpose computer 131 and a semiconductor-based microprocessor (in the form of a microchip) or a macroprocessor.

The memory 402 can include any one or combination of volatile memory elements (e.g., random-access memory (RAM, such as dynamic-RAM or DRAM, static-RAM or SRAM, etc.)) and nonvolatile-memory elements (e.g., read-only memory (ROM), hard drives, tape drives, compact-disk drives (CD-ROMs), etc.). Moreover, the memory 402 may incorporate electronic, magnetic, optical, and/or other types of storage media now known or later developed. Note that the memory 402 can have a distributed architecture, where various components are situated remote from one another, but accessible by processor 400.

The software in memory 402 may include one or more separate programs, each of which comprises an ordered listing of executable instructions for implementing logical functions. In the example of FIG. 4, the software in the memory 402 includes image manager 416 that functions as a result of and in accordance with operating system 406. Memory 402 also includes image files 510 that contain information used to produce one or more representations of diagnostic images acquired by the diagnostic image-acquisition system 110 of FIG. 1. Operating system 406 preferably controls the execution of computer programs, such as image manager 416, and provides scheduling, input-output control, file and data management, memory management, and communication control and related services.

In an embodiment, image manager 416 is one or more source programs, executable programs (object code), scripts, or other collections each comprising a set of instructions to be performed. It will be well understood by one skilled in the art, after having become familiar with the teachings of the system and method, that image manager 416 may be written in a number of programming languages now known or later developed.

The input device(s) 410 may include, but are not limited to, a keyboard, a mouse, or other interactive-pointing devices, voice-activated interfaces, or other operator-machine interfaces (omitted for simplicity of illustration) now known or later developed. The input device(s) 410 can also take the form of an image-acquisition device or a data-file transfer device (e.g., a floppy-disk drive, a digital-video disk (DVD) player, etc.). Each of the various input device(s) 410 may be in communication with the processor 400 and/or the memory 402 via the local interface 408. Data received from an image-acquisition device connected as an input device 410 or via the network interface device(s) 414 may take the form of a plurality of pixels, or a data file such as image file 510.

The output device(s) 412 may include a video interface that supplies a video-output signal to a display monitor associated with the respective general-purpose computer 131. Display devices that can be associated with the general-purpose computer 131 are conventional CRT based displays, liquid-crystal displays (LCDs), plasma displays, image projectors, or other display types now known or later developed. It should be understood, that various output device(s) 412 may also be integrated via local interface 408 and/or via network-interface device(s) 414 to other well-known devices such as plotters, printers, copiers, etc.

Local interface 408 may also be in communication with input/output devices that communicatively couple the general-purpose computer 131 to a network. These two-way communication devices include, but are not limited to, modulators/demodulators (modems), network-interface cards (NICs), radio frequency (RF) or other transceivers, telephonic interfaces, bridges, and routers. For simplicity of illustration, such two-way communication devices are represented by network interface(s) 414.

Local interface 408 is also in communication with time-code generator 430. Time-code generator 430 provides a time-varying signal to the image manager 416. The time-varying signal can be generated from an internal clock within the general-purpose computer 131. Alternatively, the time-code generator 430 may be in synchronization with an externally generated timing signal. Regardless of its source, time-code generator 430 forwards the time-varying signal that is received and applied by image manager 416 each time an image is acquired by the image-management system 120 for the first time.

When the general-purpose computer 131 is in operation, the processor 400 is configured to execute software stored within the memory 402, to communicate data to and from the memory 402, and to generally control operations of the general-purpose computer 131 pursuant to the software. The image manager 416 and the operating system 406, in whole or in part, but typically the latter, are read by the processor 400, perhaps buffered within the processor 400, and then executed.

The image manager 416 can be embodied in any computer-readable medium for use by or in connection with an instruction-execution system, apparatus, or device, such as a computer-based system, processor-containing system, or other system that can fetch the instructions from the instruction-execution system, apparatus, or device, and execute the instructions. In the context of this disclosure, a “computer-readable medium” can be any means that can store, communicate, propagate, or transport a program for use by or in connection with the instruction-execution system, apparatus, or device. The computer-readable medium can be, for example but not limited to, an electronic, magnetic, optical, electromagnetic, infrared, or semiconductor system, apparatus, device, or propagation medium now known or later developed. Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory.

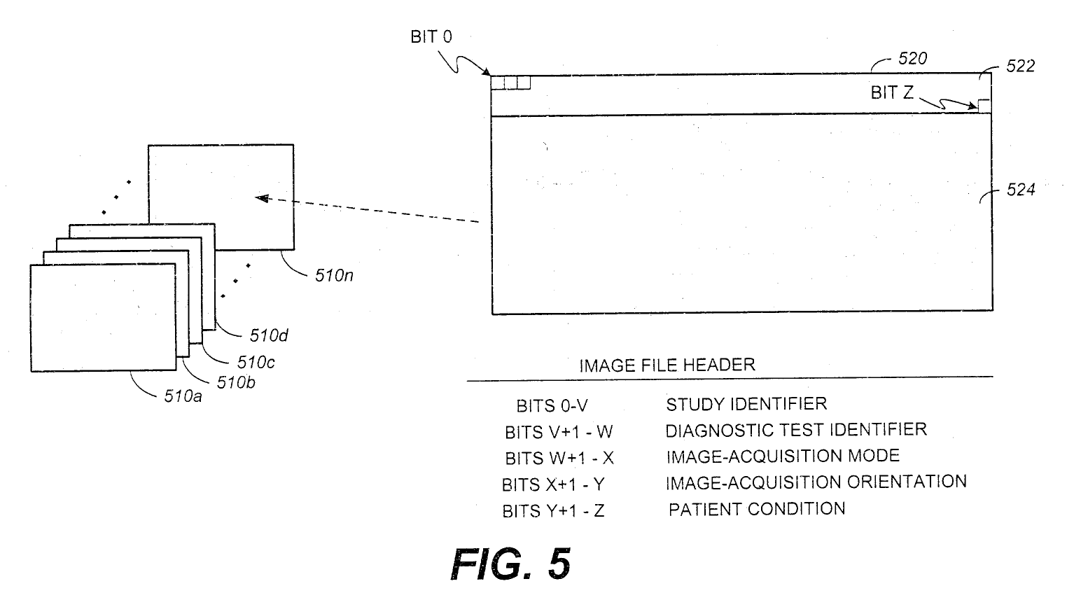

FIG. 5 presents an example of an internal data structure 520 that can be applied to one or more image files 510. As illustrated, each of the image files 510 includes an image file header 522 and image information 524. As illustrated in the table shown below the data structure 520, the image file header 522 includes a plurality of bits with bits 0 through V designated to store a study identifier, bits V+1 through W designated to store a diagnostic test identifier, bits W+1 through X designated to store an image-acquisition mode, bits X+1 through Y designated to store an image-acquisition orientation, and bits Y+1 through Z designated to store a patient condition.

Those skilled in the art should understand that the example image-file header 522 may be arranged in various ways, which include but are not limited to rearranging the order and relative length in bits of each of the image-file header parameters, adding image parameters including operational parameters associated with the underlying image-acquisition system, adding patient conditions, etc.

In an alternative embodiment (not illustrated), the first of a sequence of images may include an image-file header 522 that includes an image loop-length parameter. The image loop-length parameter identifying a number of images and/or their individual locations in memory 402 to enable a plurality of the diagnostic images to be concatenated together to permit time motion analysis of the patient's heart.

Note that the diagnostic image-acquisition system 110 can be triggered as explained above to capture diagnostic images in real-time for motion studies of the various structures of the patient-under-test's heart. Alternatively, the diagnostic image-acquisition system 110 can be triggered by various characteristics of the patient's electrocardiographic results to acquire diagnostic images at particular portions of the patient-under-test's heart cycle.

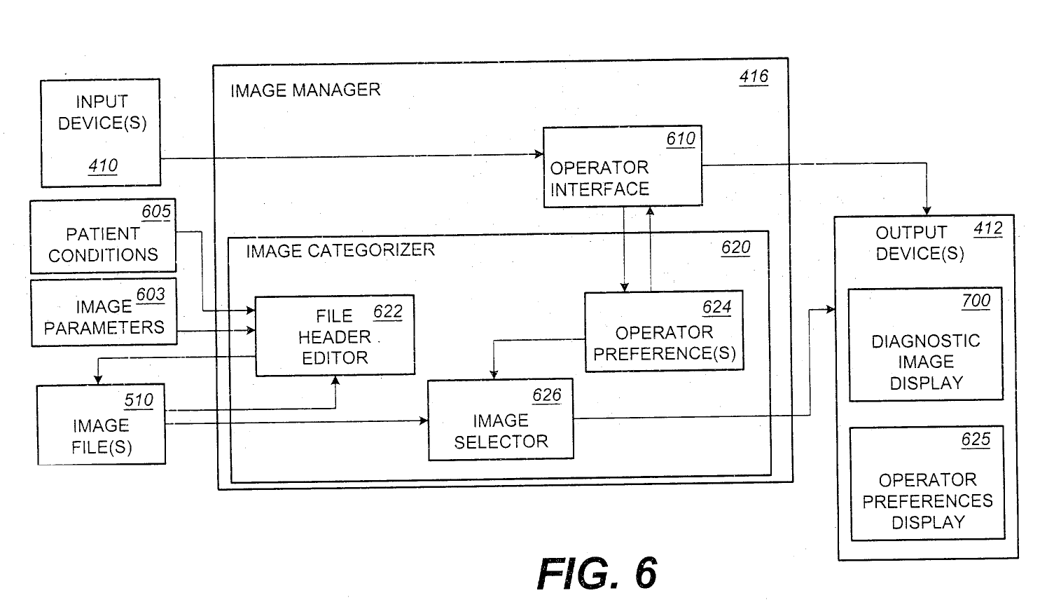

Reference is now directed to FIG. 6, which presents an embodiment of a functional block diagram of the image manager 416 of FIG. 4. As illustrated in FIG. 6, image manager 416 comprises an operator interface 610 and an image categorizer 620. Operator interface 610 is in communication with one or more input device(s) 410, the image categorizer 620, and one or more output device(s) 412. Image categorizer 620 includes a file-header editor 622, operator preferences 624, and an image selector 626.

In a first mode of operation, image manager 416 receives information indicative of an operator's preferences for observing a plurality of diagnostic images in an arrangement that provides a composite view. In this regard, operator interface 610 is configured to present an operator preferences display 625 that is arranged to provide both a summary of presently selected display preferences for viewing images of the type typically provided by a presently active diagnostic test type and a plurality of options for modifying a diagnostic image display 700. The operator preferences display 625 may also include an indication of a default display arrangement for the presently active diagnostic test type.

An operator of the image-management system 120 uses the operator interface 610 to configure one or more operator preferences 624 for each diagnostic test type that can be performed by the diagnostic image-acquisition system 110. Operator preferences 624 include information that describes the relative position and size of each of a plurality of diagnostic images identified by a combination of imaging parameters and patient conditions in addition to the diagnostic test type. Operator preferences 624 can also include information that describes various clinical data, which a diagnostician may prefer to observe when analyzing the various images.

In an image acquisition and storage mode, image categorizer 620 receives and processes each diagnostic image the first time the image is processed by the image-management system 120. As illustrated in FIG. 6, the file-header editor 622 receives image parameters 603 and patient conditions 605 and associates the various parameters and conditions as observed at the time each diagnostic image was acquired by the diagnostic image-acquisition system 110 to the various image files 510 as described above with regard to file structure 520. File-header editor 622 modifies the respective image files 510 and returns the updated image files 510 to data store 140 (FIG. 1) or an internal data-storage device associated with general-purpose computer 131 of workstation 130. Note that in some embodiments the data store 140 can be arranged to facilitate image data access. Various arrangements may include storing related images in folders.

In an image display mode, image categorizer 620 applies operator preferences 624 over a plurality of previously acquired and modified diagnostic images 510 to identify which of the plurality of images meet the preferred criteria as selected by an operating diagnostician of the image-management system 120. As illustrated in FIG. 6, image files 510 are filtered or otherwise identified by the image selector 626 in accordance with the operator specified preferences for arranging the diagnostic images. For example, a diagnostician may be interested in observing different anatomical views of a cardiac patient's heart (e.g., apical-4, apical-2, parasternal long, parasternal short, etc.) over four stages of stress. The stages of stress can be applied as described above. Alternatively, the stages of stress can be identified by dosage levels of one or more stimulants introduced into the patient-under-test's bloodstream to increase the patient's heart rate or blood flow.

In one display arrangement, the diagnostician may prefer to see the apical-4-stage IV image loop on the right side of the diagnostic-image display 700 and the apical 4-stage III image loop on the left side of the diagnostic-image display 700. The diagnostician may specifically request that the technician observe and record the stimulant and dosage levels, the patient s heart and respiratory rates, as well as other types of clinical information and/or image acquisition parameters during the examination. The diagnostician may then add the clinical information and image-acquisition parameters over a designated portion of the diagnostic-image display 700.

Image selector 626 uses timing information inserted by file header editor 622 into each of the plurality of image files 510 to synchronize the various diagnostic images that are arranged on a particular diagnostic display 700. As described above, the relative timing information may be provided by the time code generator 430 (FIG. 4) and/or the electrocardiographic transducers 215 (FIG. 2).

Alternatively, image selector 626 can be programmed to extract relative timing information from diagnostic images acquired and stored with other diagnostic imaging systems. It should be understood that various timestamps or other indication of the image acquisition time can be encoded and inserted into the image-file header 522 as described above, a separate image-management database, or may be encoded within the image information 524. In still another alternative, image selector 626 includes logic that identifies closely related image subject matter, that is, diagnostic images of structures acquired from slightly different acquisition angles.

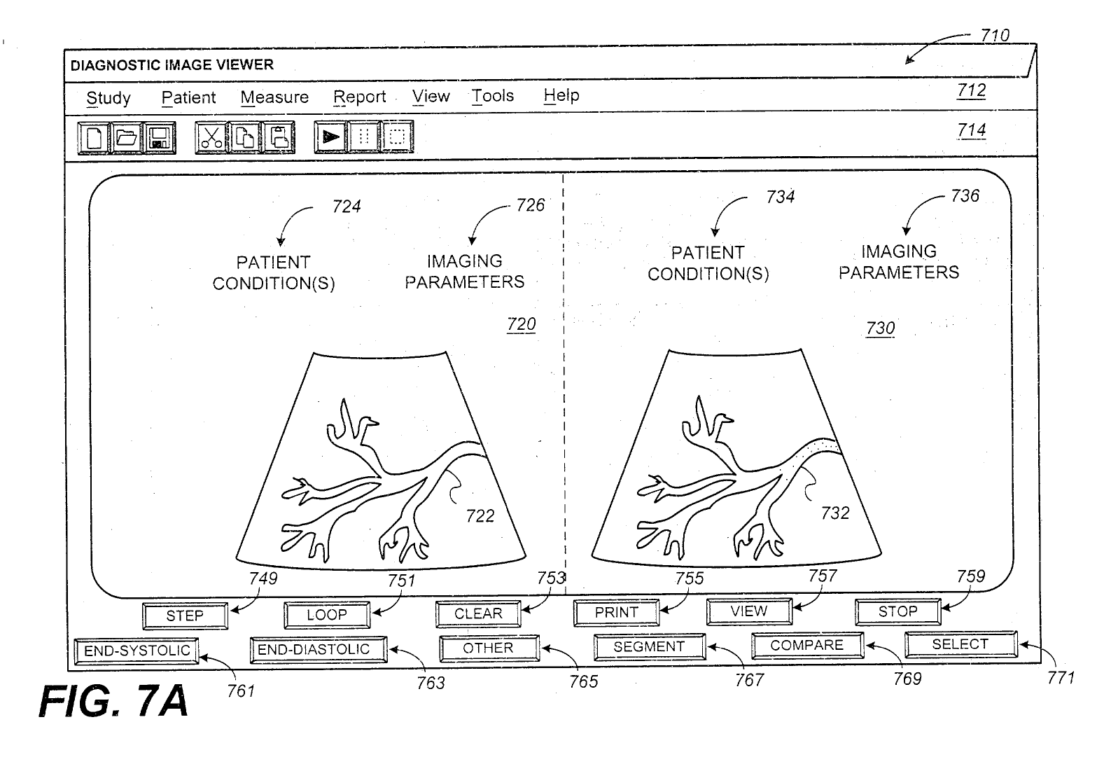

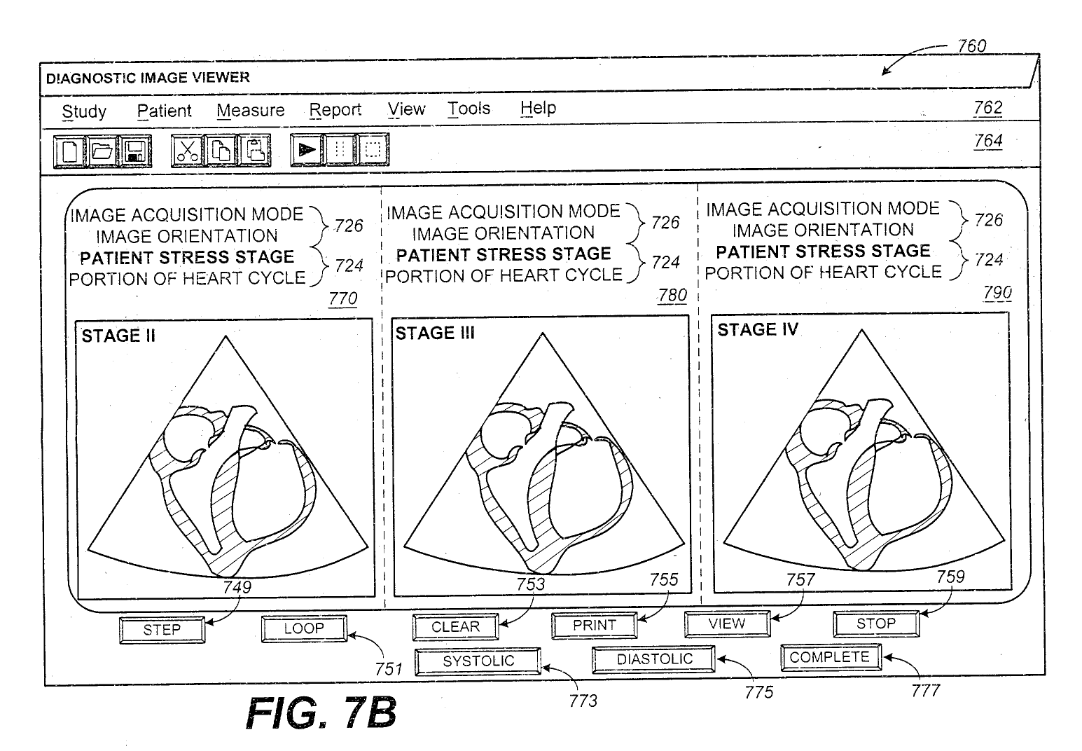

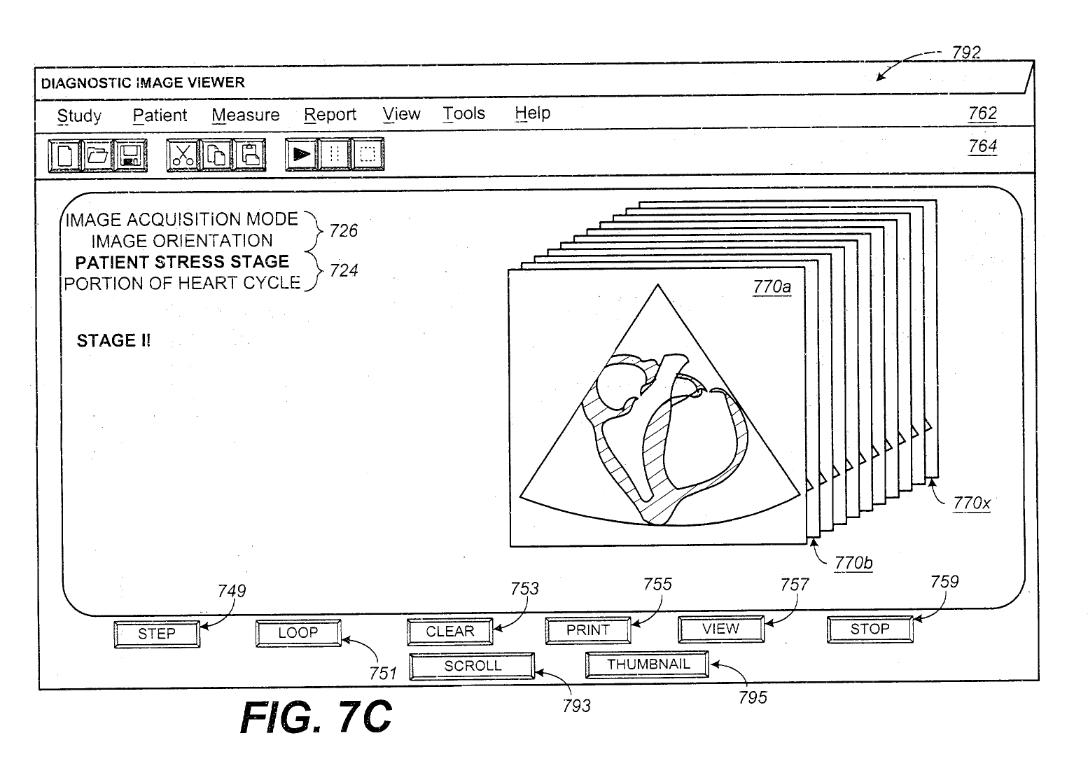

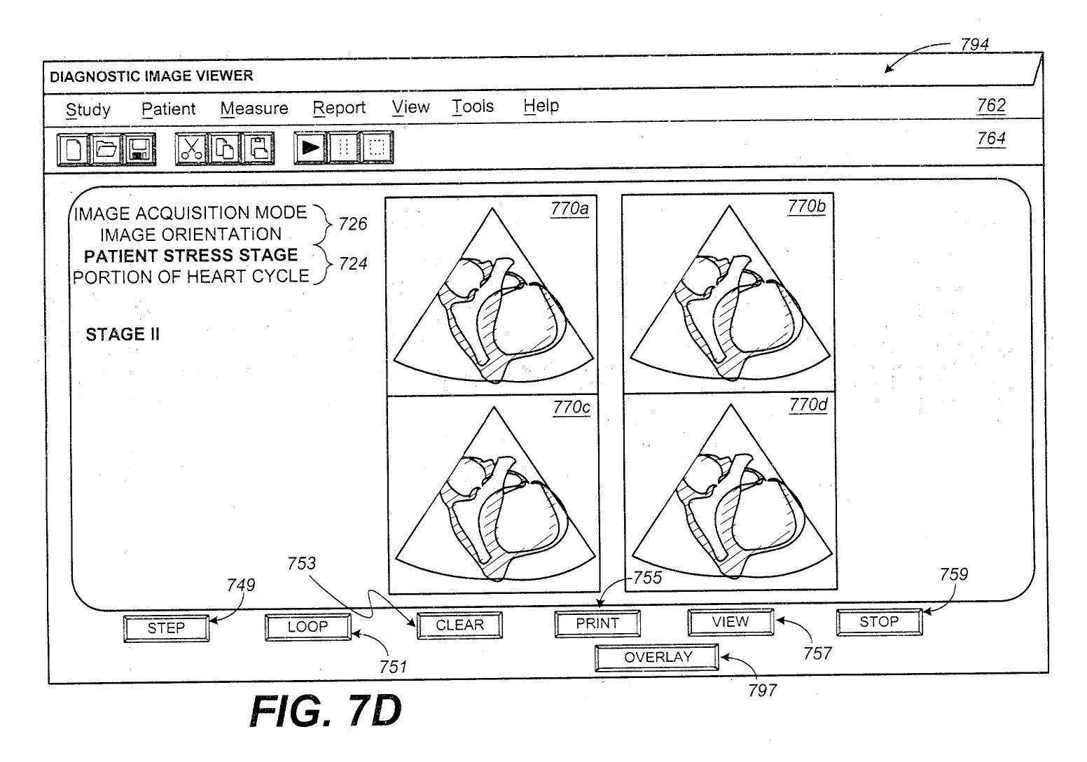

FIG. 7A illustrates an embodiment of a diagnostic image viewer 710 that can be programmed to present a plurality of diagnostic images in accordance with the observation preferences of a diagnostician of the image-management system 120. As shown in FIG. 7A, diagnostic-image viewer 710 is a graphical-user interface (GUI) that includes a pull-down menu bar 712 and a plurality of iconic task pushbuttons. The GUI includes a left-side diagnostic-image panel 720 and a right-side diagnostic-image panel 730. The left-side diagnostic-image panel 720 includes a diagnostic image of tissue(s) of interest 722 (e.g., a portion of a patient's cardiac blood supply vessels), patient conditions 724, as well as imaging parameters 726. Similarly, the right-side diagnostic-image panel 730 includes a diagnostic image of tissue(s) of interest 732 acquired after the diagnostic image presented in the left-side diagnostic-image panel 720 as can be seen by the perfusion of contrast agent in the blood supply entering the cardiac vessel from the right. Right-side diagnostic-image panel 730 also includes patient conditions 734 and imaging parameters 736 as observed when the diagnostic image of the tissue(s) of interest 732 was acquired.

Diagnostic-image viewer 710 also includes a plurality of functional pushbuttons labeled step, “loop,” “clear,” “print,” “view,” and “stop.” Step pushbutton 749 is associated with logic that displays successive diagnostic images one at a time within both the right and left-side diagnostic-image panels 730, 720, respectively, in the sequence that they were acquired during the stress examination. Loop pushbutton 751 is associated with logic that displays successive diagnostic images within both the right and left-side diagnostic-image panels 730, 720, respectively, in real-time or as triggered by various portions of the heart cycle in the sequence that they were acquired during the stress examination. Image loops are desired to observe contrast agent perfusion of the tissues of interest, which may take several cardiac cycles. Clear pushbutton 753 is associated with logic that removes the diagnostic images of the tissue(s) of interest 722, 732, patient conditions 724, 734, and imaging parameters 726, 736 from the diagnostic image viewer 710. Print pushbutton 755 is associated with logic that forwards the present condition of the diagnostic image viewer 710 to a hard-copy device of choice. View pushbutton 757 is associated with logic that enables a diagnostician to enlarge a select portion of the diagnostic images of the tissue(s) of interest 722, 732. Preferably, when the diagnostician indicates that a particular portion of one of the two diagnostic images of the tissue(s) of interest 722, 732 should be enlarged, the other diagnostic image of interest responds accordingly. Stop pushbutton 759 is associated with logic that prevents the diagnostic image viewer 710 from progressing to a subsequent set of images while in the loop display mode.

The diagnostic image viewer 710 includes additional control interfaces that enable a diagnostician to modify various preferred arrangements of the diagnostic images. The additional control interfaces include end-systolic pushbutton 761, end-diastolic pushbutton 763, other pushbutton 765, segment pushbutton 767, compare pushbutton 769, and select pushbutton 771.

End-systolic pushbutton 761 is associated with logic that identifies and displays diagnostic images acquired in synchronization with the termination of the systolic portion of the patient's heart cycle. End-diastolic pushbutton 763 is associated with logic that identifies and displays diagnostic images acquired in synchronization with the termination of the diastolic portion of the patient's heart cycle. Other pushbutton 765 is associated with logic that displays a menu that provides a mechanism for a diagnostician to select only images acquired at some other portion of the patient's heart cycle for display.

Segment pushbutton 767 is associated with logic that enables a diagnostician to divide an image loop into multiple image loops each having the same period. For example, in a default mode, the image manager 416 may be programmed to identify an image loop segment acquired during the first cardiac cycle after one or more contrast agent destructive ultrasound energy pulse(s) and identify and display other real-time image loops acquired over the same cardiac cycle. Similarly, an image loop acquired during the n^{th} cardiac cycle after the contrast agent destructive ultrasound energy pulse(s) can be arranged for display with real-time image loops acquired over the n^{th} cardiac cycle.

Compare pushbutton 769 is associated with logic that enables a diagnostician to select and display a specific cardiac cycle after the contrast agent destructive ultrasound energy pulses acquired with the patient at rest to a specific cardiac cycle acquired during a designated level of stress. Note that the cardiac cycles are not necessarily synchronized. Compare pushbutton 769 is preferrably programmed with a set of default values. In addition, compare pushbutton 769 initiates a menu or other secondary interface (e.g., a popup interface) to permit a diagnostician to controllably select multiple options when comparing segmented image loops. Diagnostic image viewer 710 includes a secondary interface (not shown) such as a pushbutton that enables a diagnostician to quickly select each preferred diagnostic imaging display.

The additional control interfaces may be used when observing real-time myocardial opacification in image loops. When comparing diagnostic images acquired in real-time, image manager 416 may be controllably adjusted to display image loops with tissue perfusion at slower rates to enable a diagnostician to observe blood flow through various tissues of interest.

With controllably triggered images, it is often desired to observe multiple images of the heart at the same portion of the cardiac cycle (e.g., end systole) with images from approximately the same trigger interval provided within the diagnostic image viewer 710. Image manager 416 is programmed with the flexibility to permit a diagnostician to compare different parts of one loop to different parts of the same loop or another loop acquired at a certain patient condition or anatomical view. For example, comparing the triggered or real-time perfusion images from a particular view every 4^{th} cardiac cycle at rest to every cardiac cycle during peak stress has shown to be extremely useful. The DIMS 100 enables the diagnostician to arrange these multiple image loops for comparison and observation automatically once the diagnostician has entered and stored the diagnostician's display preferences.

In some imaging modes, contrast agent destruction can occur with every image or frame. Consequently, image loops in these imaging modes often comprise a sequence of images where the delay between acquiring each subsequent image changes within the loop. For example, diagnostic image loops can consist of a sequence that triggers (i.e., acquires an image) every n^{th} cardiac cycle for each frame, where n can progressively increase. A typical sequence may look something like 1, 1, 1, 2, 2, 2, 4, 4, 4, 8, 8, 8 where 1, 2, 4, and 8 represents the number of complete heart cycles prior to acquiring the next subsequent image. The sequence above would take 45 heart cycles to complete and would produce 12 images.

Select pushbutton 771 is associated with logic that initiates a secondary interface that enables a diagnostician to identify one or more specific images from the triggered sequence on a frame by frame basis for comparision. A default mode selects each of the triggered images. The diagnostic image loops can then be observed to derive tissue reperfusion functions for the tissues-of-interest. The DIMS 100 could be programmed to use this original image loop with varying delays between subsequent images and create a diagnostic loop which plays back the images as if they were acquired in real-time. In this way, the DIMS 100 greatly assists a diagnostician in the task of comparing the triggered myocardial tissue opacification loops.

The various functions associated with segment pushbutton 767, compare pushbutton 769, and select pushbutton 771 are applicable to both real-time image loops as well as triggered image loops.

Those skilled in the art will understand that while the sample diagnostic-image panels in FIG. 7A are shown in a side-by-side orientation that alternative image orientations are possible. For example, a diagnostician may prefer to have paired images displayed in a vertical arrangement, or when it is desired to display various images acquired from four distinct stress stages, the operator may elect to observe the diagnostic images in a 2×2 arrangement (i.e., with a diagnostic image in each corner of the display).

FIG. 7B illustrates an alternative embodiment of a diagnostic image viewer 760 that can be programmed to present a plurality of diagnostic images in accordance with the observation preferences of a diagnostician of the image-management system 120. As shown in FIG. 7B, diagnostic-image viewer 760 is a GUI that includes a pull-down menu bar 762 and a plurality of iconic task pushbuttons 764. The GUI includes a left-side diagnostic-image panel 770, a center diagnostic-image panel 780, and a right-side diagnostic-image panel 790. The left-side diagnostic-image panel 770 includes a diagnostic image of tissue(s) of interest (e.g., a slice of a patient's heart), as well as a host of patient conditions 724 and imaging parameters 726 as observed when the respective images were acquired. As illustrated, patient conditions 724 include a patient stress stage and a portion of the patient's heart cycle. In the example, the diagnostic image of the tissue(s) of interest was observed when the patient was in stress stage II.

The center diagnostic-image panel 780 includes another image in the same image acquisition mode, image orientation, and portion of the heart cycle. The center diagnostic-image panel 780 also includes patient conditions 724 and imaging parameters 726 as observed when the respective image was acquired. In the example the diagnostic image of the tissue(s) of interest was observed when the patient was in stress stage III.

Similarly, the right-side diagnostic-image panel 790 includes another image in the same image acquisition mode, image orientation, and portion of the heart cycle as the diagnostic images in the image panels to the left. The right-side diagnostic-image panel 790 also includes patient conditions 724 and imaging parameters 726 as observed when the respective image was acquired. In the example, the diagnostic image of the tissue(s) of interest was observed when the patient was in stress stage IV.

Diagnostic-image viewer 710 also includes a plurality of functional pushbuttons labeled “step,” “loop,” “clear,” “print,” “view,” and “stop.” The various functional pushbuttons can be programmed to enable diagnostic-image viewer control with each of the respective functional pushbuttons operating as described above with regard to the GUI illustrated in FIG. 7A. It should be understood that the various functional pushbuttons provides a diagnostician flexibility when observing the various diagnostic images acquired during a patient examination.

For example, if in addition to wall motion images a technician acquires images with myocardial tissue opacification to permit a diagnostician to observe myocardial vessel perfusion of contrast agents, the diagnostician may desire several different ways to display and compare the various images. The diagnostician may want to compare heart wall motion. The diagnostician may want to compare images with myocardial tissue opacification with other like acquired images from a different view angle (i.e., a different transducer position and orientation). The diagnostician may also want to compare images with myocardial opacification with images containing heart wall motion. The image-management system 120 of the DIMS 100 enables a diagnostician to configure and store multiple diagnostic image arrangements along with the imaging parameters and patient conditions observed at the time the images were acquired. The DIMS 100 also enables a diagnostician to quickly cycle through the various choices.

Generally, diagnosticians do not prefer to view heart wall motion images in the same fashion as images containing myocardial tissue opacification. One preferred method of observing image loops of these different types of images is to have them start together and run in sequence as they were acquired. Some myocardial tissue opacification image loops are acquired in real time. Some others are controllably triggered as is the case with contrast agent destruction and observation of the tissues of interest as the blood supply reperfuses the tissues with contrast agent. With real time images, diagnosticians may desire to locate an image or frame where contrast agent destruction occurred and to define that image as the first image in the image loop. Image manager 416 is programmed to automatically define the first image in an image loop.

The diagnostic image viewer 760 includes additional control interfaces that enable a diagnostician to modify various preferred arrangements of the diagnostic images. The additional control interfaces include systolic pushbutton 773, diastolic pushbutton 775, and cycle pushbutton 777. Systolic pushbutton 773 is associated with logic that identifies and displays diagnostic images acquired in synchronization with the systolic portion of the patient's heart cycle. Diastolic pushbutton 775 is associated with logic that identifies and displays diagnostic images acquired in synchronization with the diastolic portion of the patient's heart cycle. Cycle pushbutton 777 is associated with logic that displays diagnostic images acquired over the entire heart cycle.

The additional control interfaces may be used when observing wall motion image loops. When comparing diagnostic images acquired over the systolic or diastolic portions of the patient's heart cycle, image manager 416 is programmed to synchronize selected image loops acquired over various stages of patient stress. Consequently, image loops acquired with different patient heart rates may be coordinated to start and stop with the same event in the patient's heart cycle. Synchronization of diagnostic images acquired over various stages of stress (i.e., patient heart rates) enables a diagnostician to compare tissue movement throughout the patient's heart cycle over different stages of stress.

It should also be understood that while the various examples illustrated and described above include two-dimensional images, the image manager 416 can be programmed to apply the display techniques using three-dimensional images as well. Furthermore, it should be understood that while the various control pushbuttons (e.g., pushbuttons 749 through 771) have been illustrated and described in association with the diagnostic image viewer of the general-purpose computer 131 the controls may be integrated with the DIAS 110.

FIG. 7C illustrates a way that the DIMS 100 can arrange a series of diagnostic images to provide another diagnostic perspective that may prove useful when the diagnostician is interested in a particular area of the patient's anatomy. DIMS 100 can identify and arrange a series of diagnostic images each acquired under a given stage of stress but from slightly different view angles. As illustrated in FIG. 7C the diagnostic image viewer 792 tiles diagnostic images 770a through 770x. Scroll pushbutton 793 is associated with logic that will move subsequent images in the series to the front of the stack for observation for a controllable period of time until the series of images acquired from each available view angle is complete. Thumbnail pushbutton 795 is associated with logic that creates the display mode illustrated in FIG. 7D.

As illustrated, the diagnostic image viewer 794 displays images 770a through 770d. As described above, each of the separate images include a particular view of a patient's heart with each of the views having a slightly different acquisition perspective. It should be appreciated that the number of separate thumbnail images (770a-770d shown) may vary depending upon the relative size of the display monitor to the operator desired size of the each thumbnail in the series. Overlay pushbutton 797 is associated with logic that returns to the image overlay display mode illustrated in FIG. 7C.

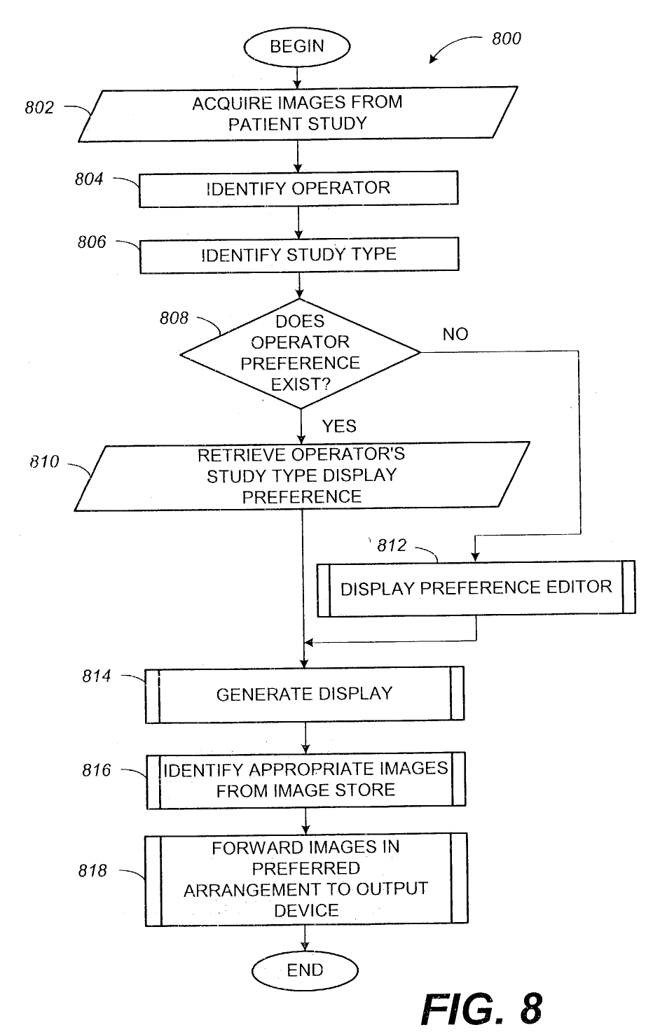

Reference is now directed to FIG. 8, which illustrates a flowchart describing a method for improved diagnostic-image displays 800 that may be implemented by the DIMS 100 of FIG. 1. As illustrated in FIG. 8, the method for improved diagnostic-image displays 800 begins with acquiring images from a patient study or examination as indicated in data operation 802. In operation 804, an operator of the DIMS 100 is identified. In operation 806, the particular diagnostic imaging test type is identified. Next, as indicated in query 808, the DIMS 100 may determine if an operator display preference has been previously stored by the identified operator for the identified study type. When an operator preference exists as indicated by the flow control arrow labeled, “YES” the DIMS 100 retrieves the operator's display preference parameters for the identified study as indicated in operation.

Otherwise, when an operator preference has not been previously identified, the DIMS 100 responds by entering the display preference editor as illustrated in operation 812. Once the diagnostician has indicated those images to be arranged and display preferences, the DIMS 100 responds by generating the display as illustrated in operation 814. The DIMS 100 also responds by identifying appropriate images from the image store as indicated in operation 816. Thereafter, as illustrated in operation 818, the DIMS 100 forwards the identified diagnostic images in the diagnosticians preferred arrangement for observing images acquired via the identified test.

It should be emphasized that the above-described embodiments of the diagnostic image-management system and its various components are merely possible examples of implementations, merely set forth for a clear understanding of the principles of the system and method for improved diagnostic image displays. Many variations and modifications may be made to the above-described embodiment(s) of the invention without departing substantially from the principles of the invention. For example, the control interfaces illustrated in FIGS. 7A-7D and described above may be integrated as physical pushbuttons, selector knobs, thumb wheel interfaces, etc. with the DIAS 110. Those skilled in the art will understand that these control interfaces may be additional and/or an alternative embodiment to the graphical-user interface(s) described above. All such modifications and variations are intended to be included herein within the scope of this disclosure and are protected by the following claims.

We claim:

1. A diagnostic-imaging management system, comprising:
means for acquiring a plurality of medical diagnostic images of a patient, wherein each of the plurality of medical diagnostic images is associated with an image-acquisition time;
means for associating an imaging parameter with each of the plurality of medical diagnostic images;
means for associating a patient condition with each of the plurality of medical diagnostic images; and
means for selectively displaying a subset of the plurality of medical diagnostic images in accordance with a directive responsive to the image-acquisition time, the imaging parameter, and the patient condition.

2. The system of claim 1, wherein the imaging parameter is selected from the group consisting of image-acquisition mode and anatomical view.

3. The system of claim 1, wherein the patient condition comprises a stress stage defined by a range of heart-cycle rates.

4. The system of claim 1, further comprising:
means for associating a loop identifier with a time-based sequence of images selected from the plurality of medical diagnostic images, wherein the time-based sequence of images share at least one parameter selected from the group consisting of imaging and patient conditions.

5. The system of claim 4, further comprising:
means for storing the time-based sequence of images.

6. The system of claim 4, further comprising:
means for segmenting the time-based sequence of images.

7. The system of claim 4, further comprising:
means for controllably generating a composite view including a plurality of images wherein each of the plurality of images is selected from a separate sequence of images.

8. The system of claim 4, wherein each of the images comprising the time-based sequence of images is associated with information relating the image-acquisition time to a concurrently acquired portion of the patient's heart cycle.

9. The system of claim 8, wherein the concurrently acquired representation of the patient's heart cycle reflects the completion of the systole.

10. The system of claim 8, wherein the concurrently acquired representation of the patient's heart cycle reflects the completion of the diastole.

11. The system of claim 4, further comprising:
means for controllably acquiring a sequence of images every n^{th} heart cycle.

12. The system of claim 11, further comprising:
means for controllably displaying images acquired every n^{th} heart cycle.

13. The system of claim 12, further comprising:
means for controllably displaying images acquired during a select heart cycle when the patient is at a first stress stage along with images acquired during a select heart cycle when the patient is at a second stress stage.

14. The system of claim 5, wherein the means for storing generates an image file comprising information selected from the group consisting of the diagnostic-test identifier, the image-acquisition time, the imaging parameter, and the patient condition.

15. The system of claim 1, wherein the means for selectively displaying further comprises means for identifying images that share a common imaging parameter.

16. The system of claim 1, wherein the means for selectively displaying further comprises means for identifying images that share a common patient condition.

17. A method for arranging a plurality of diagnostic images, comprising:
collecting a plurality of diagnostic images of a patient, wherein each of the diagnostic images is associated with an image-acquisition mode and a patient condition;
receiving a diagnostic directive comprising information responsive to a diagnostician's preference to observe diagnostic images associated with an image-identifier selected from the group consisting of image-acquisition orientation, image-acquisition mode, and patient condition;
identifying a subset of the plurality of diagnostic images responsive to the diagnostic directive; and
forwarding the subset of the plurality of diagnostic images to an output device.

18. The method of claim 17, further comprising:
aligning a composite representation of a number of diagnostic images selected from the subset of the plurality of diagnostic images.

19. The method of claim 18, wherein aligning comprises arranging the diagnostic images in accordance with an operator preference.

20. The method of claim 17, further comprising:
forming a diagnostic-image loop comprising a plurality of diagnostic images associated with the same image-acquisition mode, wherein the plurality of diagnostic images are presented in sequence.

21. The method of claim 20, further comprising:
aligning a composite representation of a number of diagnostic-image loops selected from the subset of the plurality of diagnostic images.

22. The method of claim 21, wherein aligning comprises arranging the number of diagnostic-image loops in accordance with an operator preference to observe diagnostic-image loops having the same image-acquisition orientation over multiple stages of stress.

23. The method of claim 17, wherein receiving comprises a diagnostician's preference to observe diagnostic images having the same image-acquisition orientation over multiple stages of stress.

24. The method of claim 17, wherein receiving comprises a diagnostician's preference to observe diagnostic images having the same image-acquisition orientation over multiple stages of stress.

25. The method of claim 17, wherein identifying comprises associating a plurality of diagnostic images acquired at the same portion of the patient's heart cycle.

26. The method of claim 25, wherein identifying further comprises aligning the plurality of diagnostic images in time.

27. A diagnostic-imaging system, comprising:
a patient interface configured to measure a patient condition and generate a first control signal;
an ultrasound-imaging system communicatively coupled to the patient interface, the ultrasound imaging system configured to obtain a plurality of medical diagnostic images of a patient treated with a contrast agent over time, wherein each of the plurality of medical diagnostic images is associated with an image-acquisition time;
a medical diagnostic image manager communicatively coupled to the ultrasound-imaging system, the medical diagnostic image manager configured to associate at least one imaging parameter and the patient condition with each of the plurality of medical diagnostic images;
an operator interface communicatively coupled to the ultrasound-imaging system and the medical diagnostic-image manager, the operator interface configured to receive information from an operator of the diagnostic imaging system indicative of an operator preference for spatially arranging a plurality of medical diagnostic images.

28. The system of claim 27, wherein the ultrasound-imaging system selectively applies the first control signal to acquire images at a desired portion of the patient's heart cycle.

29. The system of claim 27, wherein the ultrasound-imaging system selectively applies a second control signal to generate a transmit pulse that alters the contrast agent within the patient enabling the ultrasound-imaging system to acquire images that contain information indicative of the reperfusion of patient tissue.

30. The system of claim 27, wherein the operator interface receives an operator preference to arrange the plurality of medical diagnostic images having a particular image-acquisition orientation over multiple ranges of patient heart rates.

31. The system of claim 27, wherein the operator interface receives an operator preference to arrange the plurality of medical diagnostic images acquired at the same portion of the patient's heart cycle.

32. The system of claim 27, wherein the operator interface receives an operator preference to arrange the plurality of medical diagnostic images acquired with a particular image-acquisition mode over multiple ranges of patient heart rates.

33. The system of claim 27, wherein the operator interface receives an operator preference to arrange the plurality of medical diagnostic images acquired with a particular image-acquisition mode over a plurality of medical diagnostic images having multiple image-acquisition orientations.

34. The system of claim 27, further comprising:
a rendering device communicatively coupled to the diagnostic image manager configured to display the plurality of medical diagnostic images in accordance with the operator preference.

35. The system of claim 34, wherein the rendering device is configured to present a plurality of diagnostic image loops in accordance with the operator preference.

36. The system of claim 35, wherein the medical diagnostic image manager is configured to present a wall motion loop coinciding with the patient's heart cycle, the loop chosen to coincide with the group consisting of the systolic portion, the diastolic portion, and the entire heart cycle of the patient.

37. The system of claim 36, wherein the medical diagnostic image manager is configured to synchronize a plurality of wall motion loops acquired over multiple stages of stress such that the loops begin and end at the same time.

38. The system of claim 35, wherein the medical diagnostic image manager is configured to present a wall motion loop coinciding with the patient's heart cycle, the loop chosen to coincide with the group consisting of the systolic portion, the diastolic portion, and the entire heart cycle of the patient.

39. The system of claim 33, wherein the medical diagnostic image manager is configured to present the plurality of images as separate thumb nail representations.

40. The system of claim 33, wherein the medical diagnostic image manager is configured to present the plurality of images in a stack.

41. The system of claim 40, wherein the operator interface receives an operator preference to alternatively observe each of the plurality of images from the stack.

42. A computer-readable medium having processor-executable instructions thereon which, when executed by a processor, direct the processor to:
apply an input indicative of an operator preference for a spatial arrangement of a plurality of subsets of medical diagnostic images acquired during an examination, wherein each of the plurality of medical diagnostic images are associated with an image-acquisition time, an imaging parameter, and a patient condition;
determine which of the plurality of medical diagnostic images match the operator preference for respective positions for observation on an output device; and
forwarding the plurality of medical diagnostic images in sequence in accordance with the associated image-acquisition time to a display device communicatively coupled to the processor.

43. The computer-readable medium of claim 42, wherein the input indicative of the operator preference identifies an imaging parameter selected from the group consisting of image-acquisition orientation and imaging mode.

44. The computer-readable medium of claim 42, wherein the input indicative of the operator preference identifies a patient condition related to the patient's heart function.

45. The computer-readable medium of claim 44, wherein the patient condition comprises a range of heart-cycle rates.

46. The computer-readable medium of claim 42, wherein the image-acquisition time is synchronized to enable simultaneous presentation of the plurality of subsets of medical diagnostic images.

47. The computer-readable medium of claim 46, wherein the image acquisition time is synchronized in accordance with real time.

48. The computer-readable medium of claim 46, wherein the image acquisition time is synchronized in accordance with events in the patient's heart cycle.
